(19) United States
(12) Patent Application Publication
(10) Pub. No.: US 2006/0211098 A1
(43) Pub. Date: Sep. 21, 2006

(54) Cells overexpressing lipoyl-protein ligase b-gene for fermentative production of r-alpha-liponic acid

(76) Inventor: Tobias Dasseler, Munchen (DE)

Correspondence Address: WILLIAM COLLARD;COLLARD & ROE, P.C., 1077 NORTHERN BOULEVARD, ROSLYN, NY, 11576, US

(21) Appl. No.: 10/529,590

(22) PCT Filed: Sep. 25, 2003
(86) PCT No.: PCT/EP03/10687

§ 371 Date: Mar. 29, 2005

(30) Foreign Application Priority Data
Oct. 2, 2002 (DE) 102459932

(51) Int. Cl. C12P 17/00; C12N 1/21; C12N 15/74

(52) U.S. Cl. 435/117; 435/252.33; 435/488

(57) ABSTRACT

The invention relates to cells and to a method for the production of R-α-liponic acid by fermentation. The inventive host organism strain, which is suitable for fermentative production of R-α-liponic acid, is characterized in that it overexpresses a gene coding for lipoyl-protein ligase B and in that it releases the formed R-α-liponic acid in free form into the culture medium.

The present invention relates to cells secreting R-α-lipoic acid and to a method for fermentative production of said R-α-lipoic acid using said cells.

R-α-Lipoic acid is an essential cofactor of particular multienzyme complexes in a multiplicity of pro- and eukaryotes. R-α-Lipoic acid is bound, in each case covalently, to the ε-amino group of a specific lysine residue of the appropriate enzyme. In this way, R-α-lipoic acid is part of the E2 subunit of pyruvate dehydrogenase (PDH) [EC 2.3.1.12] and of α-ketoglutarate dehydrogenase (KGDH) [EC 2.3.1.61] and plays an important part there as redox partner and acyl group donor in oxidative decarboxylation of α-keto acids. Moreover, lipoic acid acts as aminomethyl carrier in glycine cleavage enzyme systems.

α-Lipoic acid is an optically active molecule having a center of chirality on the C6 carbon atom. The R configuration of α-lipoic acid is the naturally occurring enantiomer. Only this form is physiologically active as cofactor of the corresponding enzymes. α-Lipoic acid may occur both in an oxidized (5-[1,2]-dithiolan-3-yl-pentanoic acid) and in a reduced form (6,8-dimercaptooctanoic acid). The term “α-lipoic acid” means hereinbelow both forms and the particular salts of α-lipoic acid, such as, for example, the calcium, potassium, magnesium, sodium or ammonium salt.

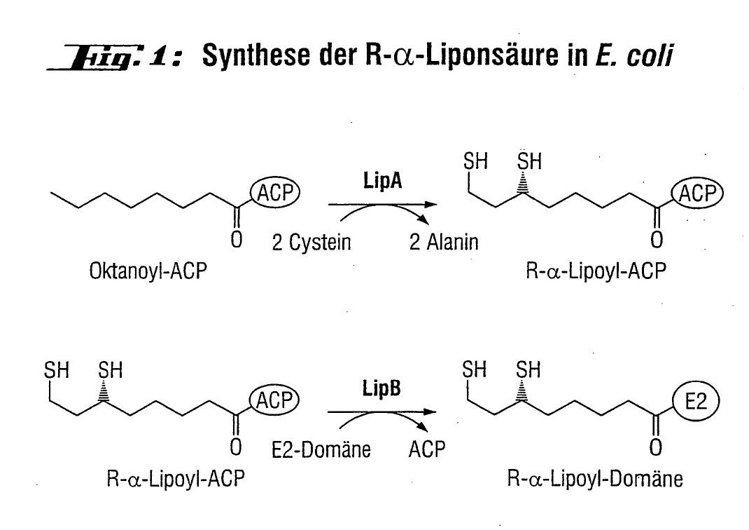

The biosynthesis of R-α-lipoic acid has been studied particularly intensively on the bacterium Escherichia coli (see FIG. 1). Here, octanoic acid which is covalently bound to the acyl-carrier protein (ACP) serves as specific precursor in lipoic acid synthesis. In a complex reaction, two sulfur atoms are transferred to the thus activated octanoic acid (Octanoyl-ACP), giving R-α-lipoyl-ACP. This reaction is catalyzed by the sulfur transferase lipoic acid synthase [EC 2.8.1.-], the lipA gene product. Serving as sulfur donor is ultimately the amino acid L-cysteine. Subsequent transfer of R-α-lipoic acid from R-α-lipoyl-ACP to the E2 subunit of the α-keto acid dehydrogenases is catalyzed by lipoyl-protein ligase B [EC 6.-.-.-], the lipB gene product, without, however, R-α-lipoyl-ACP or R-α-lipoic acid appearing as free intermediates (Miller et al., 2000, Biochemistry 39:15166-15178).

Little is known about R-α-lipoic acid biosynthesis in eukaryotes. It is assumed, however, that R-α-lipoic acid synthesis and transfer to the corresponding enzymes take place in the mitochondria of eukaryotic cells in a manner similar to that in bacteria.

Apart from its relevance as essential component of enzymes having a central role in metabolism, the importance of α-lipoic acid to pharmacotherapy and as a food supplement (Nutraceutical) was recognized already early on: owing to its two thiol groups, α-lipoic acid has a distinctive antioxidative activity and can thus protect the organism against harmful processes induced by oxidative stress. Moreover, α-dihydrolipoic acid, the reduced form of α-lipoic acid, is capable of regenerating directly or indirectly other oxidized natural antioxidants in the body, such as ascorbic acid or α-tocopherol, or also, in the case of a lack thereof, of replacing said antioxidants, owing to its property as a strong reducing agent. Accordingly, α-lipoic acid is of central importance in acting together with ascorbic acid, α-tocopherol and glutathione, the “network of antioxidants”. α-Lipoic acid is also employed in the prevention and control of type II diabetes mellitus and the damaging secondary effects thereof such as, for example, polyneuropathy, cataract or cardiovascular conditions.

Currently, the different biological activity of the two α-lipoic acid enantiomers is the subject of intensive studies, although there is more and more evidence coming to light of application of the pure R enantiomer of α-lipoic acid having distinct advantages, compared to the S form. Thus, it was shown in an in vitro experiment that only the natural R-α-lipoic acid leads to the formation of functional α-keto acid dehydrogenases. In contrast, the S enantiomer even had an inhibiting effect on stimulation of the enzyme activity by R-α-lipoic acid. The reduction of α-lipoic acid and thus regeneration of the antioxidatively active α-dihydrolipoic acid in the mitochondria are thus of essential importance to the cell. The activity of mammalian mitochondrial NADH-dependent lipoamide reductase is almost 20 times higher in combination with the R enantiomer than with the S form. In addition, R-α-lipoic acid has, compared to the S enantiomer, a distinctly stronger action on insulin-mediated glucose uptake and glucose metabolism of skeletal muscle cells of insulin-resistant rats. Moreover, the R form exhibited in an animal experiment antiphlogistic action, while the S form had rather an analgetic action. In order to avoid undesired side effects, it is therefore extremely desirable to administer α-lipoic acid in each case only in the enantiomerically pure form.

Currently, industrial production of α-lipoic acid is carried out exclusively by means of chemical methods, with the final product formed being always the racemate of R form and S form (Yadav et al., 1990, J. Sci. Ind. Res. 49: 400-409). To obtain enantiomerically pure R-α-lipoic acid, various methods have been developed. It is possible, for example, to resolve the racemate of α-lipoic acid or of one of the synthesis intermediates either chemically by means of chiral auxiliaries (Walton et. al, 1954, J. Amer. Chem. Soc. 76: 4748; DE 4137773) or enzymically (Adger et al., 1995, J. Chem. Soc., Chem. Commun.: 1563-1564). In other methods, the formation of a racemate is prevented owing to an enantioselective synthesis step, it being possible to introduce the new center of chirality either chemically (DE 3629116; DE 19533881; Bringmann et al., 1999, Z. Naturforsch. 54b: 655-661; DE 10036516) or by stereospecific biotransformation by means of microorganisms (Gopalan and Jacobs, 1989, Tetrahedron Lett. 30: 5705-5708; Dasaradhi et al., 1990, J. Chem. Soc., Chem. Commun.: 729-730; DE 10056025). Other processes, in turn, start chemical synthesis of enantiomerically pure α-lipoic acid by using a naturally occurring chiral reactant such as, for example, S-maleic acid or D-mannitol (Brookes and Golding, 1988, J. Chem. Soc. Perkin Trans. I: 9-12; Rama Rao et al., 1987, Tetrahedron Lett. 28, 2183-2186). Due to partly complicated synthesis steps, low yields and high material costs, all known methods for producing enantiomerically pure R-α-lipoic acids are currently not economical.

These days, many low molecular weight natural substances such as, for example, antibiotics, vitamins or amino acids, are frequently produced industrially by means of a fermentative method using various strains of microorganisms.

The application to the Deutschen Patent-und Markenamt, file number 10235270.4, describes cells which secrete enantiomerically pure R-α-lipoic acid and a method in which enantiomerically pure R-α-lipoic acid is produced exclusively in a fermentation process. Overexpression of a lipoic acid-synthase gene causes the cells to secrete free R-α-lipoic acid into the culture medium, but to a still very limited extent.

Only in rare cases does a single genetic manipulation in the course of the “metabolic engineering” of a wild-type strain result in overproduction of the desired compound in sufficient amounts.

Accordingly, it is the object of the present invention to provide effective cells which secrete enantiomerically pure R-α-lipoic acid into a culture medium.

This object is achieved by cells which overexpress a lipoyl protein ligase B gene (lipB gene).

The lipB gene-encoded enzyme activity here means the lipoyl protein ligase activity of a cell, which has a strong preference for R-α-lipoyl-ACP over free R-α-lipoic acid as substrate (see FIG. 1).

Overexpression in accordance with the present invention preferably means expression of the lipoyl protein ligase B gene is higher by at least a factor of 2, preferably by at least a factor of 5, compared to the particular wild type cell from which lipoyl protein ligase B gene has been isolated.

The lipoyl protein ligase B gene is preferably a gene having the sequence SEQ ID NO: 1 or a functional variant of said gene.

A functional variant in accordance with the present invention means a DNA sequence which is derived from the sequence depicted in SEQ ID NO: 1 by deletion, insertion or substitution of nucleotides, with the enzymic activity of the lipoyl protein ligase B encoded by the gene being retained.

In order to overexpress the lipB gene in the cell, a cell may have an increased lipB gene copy number and/or increased lipB gene in the expression, preferably due to suitable promoters.

Overexpression of a lipB gene increases the cellular lipoyl protein ligase B activity by in each case at least the same factor.

Preferably, a cell of the invention overexpresses a lipoyl protein ligase B gene coding for a protein comprising the sequence ID NO: 2 or functional variants having a sequence homology to SEQ ID NO: 2 of more than 40%.

The sequence homology to SEQ ID NO: 2 is preferably more than 60%, and particularly preferably more than 80%.

In the present invention, all of the homology values mentioned refer to results obtained using the BESTFIT algorithm (GCG Wisconsin Package, Genetics Computer Group (GCG) Madison, Wis.).

The copy number of a lipB gene in a cell can be increased using methods known to a skilled worker. Thus it is possible, for example, to clone a lipB gene into a plasmid vector having multiple copies per cell (e.g. pUC19, pBR322, pACYC184 in the case of Escherichia coli) and to introduce said gene into the cell. Alternatively, multiple copies of a lipB gene can be integrated into the chromosome of a cell. Integration methods which may be used are the known systems using temperate bacteriophages, integrative plasmids or integration via homologous recombination (e.g. Hamilton et al., 1989, J. Bacteriol. 171: 4617-4622).

Preference is given to increasing the copy number by cloning a lipB gene into a plasmid vector under the control of a promoter. Particular preference is given to increasing the copy number in Escherichia coli by cloning a lipB gene into a pBAD derivative such as, for example, pBAD-GFP (Crameri et al., 1996, Nat. Biotechnol. 14: 315-319). The invention therefore also relates to a plasmid which contains a lipB gene under the functional control of a promoter.

It is possible for the natural promoter and operator region of the lipB gene to serve as control region for expression of a plasmid-encoded lipB gene, but expression of a lipB gene may also be increased in particular by means of other promoters. Appropriate promoter systems which make possible either continuous or controlled inducible expression of the lipoyl protein ligase B gene, such as, for example, the constitutive GAPDH promoter of the gapA gene or the inducible lac, tac, trc, lambda, ara or tet promoters in Escherichia coli, are known to the skilled worker (Makrides S. C., 1996, Microbiol. Rev. 60: 512-538). Such constructs may be used in a manner known per se on plasmids or chromosomes.

In a particularly preferred embodiment a plasmid, which already contains a promoter for enhanced expression, such as, for example, the inducible arabinose promoter/repressor system of Escherichia coli is used for cloning a lipB gene.

Furthermore, expression may be enhanced by the presence of translational start signals, such as, for example, the ribosomal binding site or the start codon of the gene, in optimized sequence on the particular construct or by replacing codons which are rare according to the “codon usage” with more frequently occurring codons.

Cells according to the invention preferably contain a plasmid comprising a lipB gene and said modifications of the regulatory signals. In a particularly preferred embodiment, the native weak start codon of the lipB gene (TTG) is replaced by the strong start codon ATG.

A lipB gene is cloned into a plasmid vector, for example, by specifically amplifying a lipB gene by means of the polymerase chain reaction using specific primers which encompass the complete lipB gene and subsequent ligation with vector DNA fragments.

Cells of the invention whose expression of a lipB gene is increased compared to a starting cell and which have, in connection therewith, increased lipoyl protein ligase B activity may be generated from a starting cell by using standard molecular-biological techniques.

Lipoyl protein ligase B genes have been identified in a multiplicity of cells. Thus, cells of the invention may be produced preferably from cells of pro- or eukaryotic organisms, which are capable of synthesizing R-α-lipoic acid themselves (starting cell), which are accessible to recombinant methods and which are culturable by fermentation. Thus, plant or animal cells which can be grown in cell culture are also suitable for preparing cells of the invention.

Cells of the invention are preferably microorganisms such as, for example, yeast or bacteria strains. Particular preference is given to bacterial strains of the family Enterobacteriaceae, very particularly to strains of the species Escherichia coli.

Particularly suitable starting cells are also those cells which already have an increased lipoic acid-synthase activity, due to enhanced expression of the lipA gene.

A common transformation method (e.g. electroporation) is used for introducing the lipB-containing plasmids into a starting cell and selecting for plasmid-harboring clones, for example by means of resistance to antibiotics.

The invention therefore also relates to methods for preparing a cell of the invention, which comprise introducing a plasmid of the invention into a starting cell.

Another object of the invention was to provide a fermentation method which enables enantiomerically pure R-α-lipoic acid to be produced.

This object is achieved by a method which comprises culturing a cell of the invention in a culture medium, said cell secreting enantiomerically pure R-α-lipoic acid in free form into the culture medium and said enantiomerically pure R-α-lipoic acid being removed from said culture medium.

R-α-lipoic acid can be recovered from the culture medium by methods known to the skilled worker, such as centrifugation of the medium to remove the cells and by subsequent extraction or precipitation of the product.

Physiological and biochemical data indicate that lipoic acid is present in wild type cells virtually always in bound form, since R-α-lipoic acid is already synthesized in an entirely protein-bound manner (cf. FIG. 1) (Herbert and Guest, 1975, Arch. Microbiol. 106: 259-266; Miller et al., 2000, Biochemistry 39:15166-15178). Surprisingly, however, it was found within the framework of the present invention that overexpression of a lipoyl protein ligase B gene leads to the accumulation of free enantiomerically pure R-α-lipoic acid in the culture medium of the host organism. This, in turn, allows simple isolation of the product from the culture medium, after the biomass has been removed, without the need for disrupting the cells beforehand or for removing said R-α-lipoic acid from the carrier protein bound thereto (ACP or the E2 subunit of α-keto acid dehydrogenases) by means of a complicated and costly hydrolysis step.

The inventive cells for producing R-α-lipoic acid are preferably cultured in a minimal salt medium known from the literature (Herbert and Guest, 1970, Meth. Enzymol. 18A, 269-272).

In principle, it is possible to use as carbon sources any utilizable sugars, sugar alcohols or organic acids. It is furthermore possible to add short-chain fatty acids having a chain length of C2-C8, preferably having a chain length of C6-C8 (hexanoic and octanoic acid, respectively) to the medium as specific precursors for α-lipoic acid synthesis. The concentration of the carbon source added is preferably 1-30 g/l.

The cells of the invention are preferably incubated under aerobic culturing conditions and within the range of the growth temperature optimal for the particular cells over a period of 16-150 h.

Preference is given to an optimal temperature range of 15-55° C. Particular preference is given to a temperature between 30 and 37° C.

R-α-Lipoic acid produced in the method of the invention is detected and quantified, for example, by means of a bioassay using an indicator strain auxotrophic for lipoic acid (lipA mutant). This type of turbidimetric quantification of R-α-lipoic acid is known from the literature (Herbert and Guest, 1970, Meth. Enzymol. 18A, 269-272). The indicator strain used within the framework of the present invention, W1485lip2 (ATCC 25645), however, would also grow without supplemented R-α-lipoic acid, if the medium also contained acetate and succinate in addition to glucose. In order to prevent false-positive growth of said indicator strain in the bioassay when determining the R-α-lipoic acid produced, which growth is due, for example, to introduction of glucose and of acetic and succinic acid secreted by the producer strain in addition to R-α-lipoic acid, even the R-α-lipoic acid producer is grown preferably with succinate as sole carbon source. Said strain is supplemented with the supernatant of a cell culture of the invention; it is then possible to determine the lipoic acid content of the culture medium on the basis of indicator strain growth.

The examples below serve to further illustrate the invention. The bacterial strain Escherichia coli W3110/pBAD-lipB used for carrying out said examples have been deposited according to the Budapest Treaty with the DSMZ (Deutsche Sammlung für Mikroorganismen und Zellkulturen GmbH, D-38142 Braunschweig, Germany) under the number DSM 15180.

### EXAMPLE 1

### pBAD-lipB Vector Construction

A. lipB Gene Amplification

The E. coli lipB gene was amplified by means of the polymerase chain reaction (PCR) using the Pwo DNA polymerase according to common practice known to the skilled worker. The template used was the chromosomal DNA of E. coli W3110 (ATCC 27325) wild type strain. The primers used were the 5′-phosphorylated oligonucleotides lipB-fwd and lipB-rev having the following sequences:

lipB-fwd: (SEQ ID NO: 3)
5′- CAC GGA GAT GCC CAT ATG TAT CAG GAT AAA ATT
C -3′
NdeI
lipB-rev: (SEQ ID NO: 4)
5′- ATT GGG CCA TTG ATG TAT GGA ATT AAG CGG -3′

The approx. 0.68 kb DNA fragment obtained in the PCR was then purified by means of a DNA adsorption column of the QIAprep spin miniprep kit (Qiagen, Hilden, Germany) according to the manufacturer's instructions.

B. Cloning of the lipB Gene into the pKP477 Vector

A cleavage site for NdeI restriction endonuclease (recognition sequence underlined in the oligonucleotide) was introduced via the primer sequences into the PCR fragment. The purified PCR fragment was cleaved by NdeI restriction endonuclease under the conditions indicated by the manufacturer, subsequently fractionated on an agarose gel and then isolated from said agarose gel by means of the GENECLEAN kit (BIO 101 Inc., La Jolla, Calif.) according to the manufacturer's instructions.

The cloning and expression vector pKP477 was obtained from the vector PBAD-GFP (Crameri et al., 1996, Nat. Biotechnol. 14: 315-319), a derivative of the vector pBAD18, as follows: first, the GFP gene was removed by restriction of the vector PBAD-GFP with the endonucleases NheI and EcoRI. The 5′-protruding ends of the remaining, approx. 4.66 kb vector fragment were then filled in with the Klenow enzyme and the vector was finally religated using T4 ligase. E. coli cells of the strain DH5α were transformed with the ligation mixture by means of electroporation in a manner known to the skilled worker. The transformation mixture was applied to LB-ampicillin agar plates (10 g/l tryptone, 5 g/l yeast extract, 10 g/l NaCl, 15 g/l agar, 100 mg/l ampicillin) and incubated at 37° C. overnight. The desired transformants were identified by restriction analysis, after isolating the plasmids by means of a QIAprep Spin Miniprep Kit (Qiagen, Hilden, Germany). The vector obtained in this way is referred to as pKP476.

In order to remove the second NdeI cleavage site of the vector pKP476, which is located close to the origin of replication, said vector pKP476 was first partially restricted with NdeI in a manner known to the skilled worker. The linearized, i.e. only singly cut, vector fragment was isolated as described above. Subsequently, the 5′-protruding ends of this fragment were filled in using the Klenow enzyme and the vector was, as described above, religated, transformed and checked by means of restriction analysis. The thus produced plasmid pKP477 now contains the single NdeI cleavage site at an optimal distance from an optimized ribosomal binding site.

The plasmid pKP477 contains various genetic elements which allow controlled expression of any gene. It is a vector which has an origin of replication derived from the pBR plasmid family. Expression of the cloned gene is repressed by the AraC repressor and can be induced by arabinose.

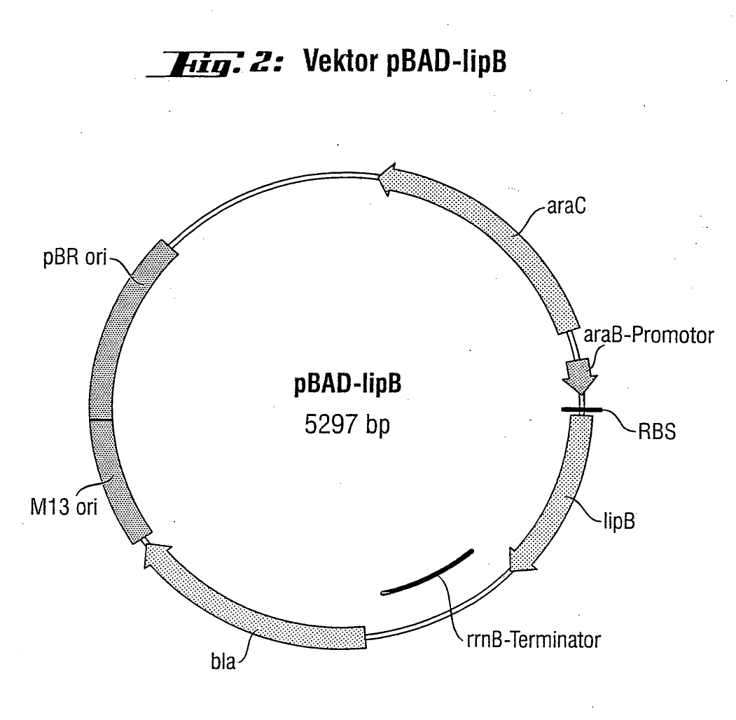

The lipB gene was cloned by cleaving the vector pKP477 with the restriction enzymes NdeI and SmaI under the conditions indicated by the manufacturer, subsequently dephosphorylating the 5′ ends of said vector by treatment with alkaline phosphatase and then purifying said vector like the lipB PCR fragment by means of the GENECLEAN method. Ligation of the PCR fragment with the cleaved and dephosphorylated vector pKP477, transformation and checking of the transformants were carried out as described above. The resulting plasmid is referred to as pBAD-lipB (FIG. 2).

### EXAMPLE 2

### Preparation of an R-α-Lipoic Acid Producer

The pBAD-lipB plasmid described in example 1 was transformed into E. coli W3110 by means of electroporation and, after selection on LB agar plates containing 100 mg/l ampicillin, the plasmid was reisolated from one of the transformants, cleaved with restriction endonucleases and checked. The control plasmid, pKP477, was treated in a similar manner.

### EXAMPLE 3

### Fermentative Production of R-α-Lipoic Acid

The strain W3110/pBAD-lipB was used for fermentative production of R-α-lipoic acid. For comparison, the strain W3110 containing the “empty” pKP477 control plasmid was cultured under exactly the same conditions.

As preculture for producer cultivation, first 5 ml of LB liquid medium containing 100 mg/l ampicillin were inoculated with the respective strain and incubated at 37° C. and 160 rpm on a shaker for 16 h. The cells were then harvested by centrifugation and washed twice with the corresponding volume of sterile saline (0.9% NaCl). The cells prepared in this way were finally used to inoculate 15 ml of BS medium (7 g/l K₂HPO₄; 3 g/l KH₂PO₄; 1 g/l (NH₄)₂SO₄; 0.1 g/l MgSO₄×7H₂O; 0.5 g/l Na₃ citrate×3H₂O; 0.2% acid-hydrolyzed casein (vitamin-free); 13.5 g/l Na₂ succinate×6H₂O; pH adjusted to 6.8 with HCl) which additionally contains 100 mg/l ampicillin, in a ratio 1:100. The producer cultures were incubated on a shaker at 37° C. and 160 rpm for 24 h. Expression of the lipoyl protein ligase B gene was induced by adding 0.2 g/l L-arabinose after approx. 4 h of incubation. After 24 h, samples were taken and the cells were removed from the culture medium by centrifugation. The R-α-lipoic acid contained therein was quantified by means of the known turbidimetric bioassay (Herbert and Guest, 1970, Meth. Enzymol. 18A, 269-272). Table 1 shows the free R-α-lipoic acid contents achieved in the particular culture supernatant after 24 h of incubation:

TABLE 1

| Strain | R-α-lipoic acid [μg/l] |
|---|---|
| W3110/pBAD-lipB | 24 |
| W3110/pKP477 | 0 |

1. A cell secreting the enantiomerically pure R-α-lipoic acid into a culture medium, wherein said cell overexpresses a lipoyl protein ligase B gene (lipB gene).

2. The cell as claimed in claim 1, wherein its expression of the lipoyl protein ligase B gene is increased by at least a factor of 2, preferably by at least a factor of 5, compared to a wild type cell from which said lipoyl protein ligase B gene has been isolated.

3. The cell as claimed in claim 1, wherein the lipoyl protein ligase B gene is a gene having the sequence SEQ ID NO: 1 or a functional variant of said gene.

4. The cell as claimed in claim 1, wherein it has an increased lipB-gene copy number or, preferably due to a suitable promoter, increased lipB-gene expression.

5. The cell as claimed in claim 1, wherein the lipoyl protein ligase B gene codes for a protein comprising the sequence ID NO: 2 or functional variants whose sequence homology to SEQ ID NO: 2 is more than 40%.

6. The cell as claimed in claim 1, wherein it is a microorganism such as, for example, a yeast or bacterial strain.

7. The cell as claimed in claim 6, wherein it is a bacterial strain of the family Enterobacteriaceae, very particularly preferably a strain of the species Escherichia coli.

8. A plasmid, wherein it contains a lipB gene under the functional control of a promoter.

9. A method for preparing a cell of the invention, wherein it comprises introducing a plasmid of the invention into a starting cell.

10. The method as claimed in claim 9, wherein the starting cell used is a cell of a prokaryotic or eukaryotic organism, which is capable of synthesizing R-α-lipoic acid, which is accessible to a recombinant method and which is culturable by fermentation.

11. A method for producing enantiomerically pure R-α-lipoic acid, which comprises culturing a cell as claimed in claim 1, in a culture medium, said cell secreting enantiomerically pure R-α-lipoic acid in free form into the culture medium and said enantiomerically pure R-α-lipoic acid being removed from said culture medium.

12. The method as claimed in claim 11, wherein the enantiomerically pure R-α-lipoic acid is removed by centrifuging the culture medium followed by extracting or precipitating said R-α-lipoic acid.

13. The method as claimed in claim 11, wherein the cells are incubated in a minimal salt medium as culture medium under aerobic culturing conditions and within the range of the optimum growth temperature for the particular cells over a period of 16-150 h.
